(19) United States
(12) Patent Application Publication
(10) Pub. No.: US 2007/0239140 A1
(43) Pub. Date: Oct. 11, 2007

(54) Controller system for crossing vascular occlusions

(75) Inventors: Victor Chechelski, Mountain View, CA (US); Gerardo V. Noriega, Mountain View, CA (US); Keith Riordan, Saratoga, CA (US); Menahem Nassi, Palo Alto, CA (US); Michael Carley, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ReVascular Therapeutics Inc., Sunnyvale, CA (US)

(21) Appl. No.: 11/636,388

(22) Filed: Dec. 7, 2006

Related U.S. Application Data
(63) Continuation-in-part of application No. 11/388,251, filed on Mar. 22, 2006.

(51) Int. Cl. A61B 17/00; A61M 31/00

(52) U.S. Cl. 606/1; 604/95.01

(57) ABSTRACT

A controller system includes a device configured to cross an occlusion or stenosis and a control unit. The occlusion-crossing device has an axial lumen and a drive shaft extending through the axial lumen. The control unit is coupled to the device. The control unit has a processor which produces a variable sound and a variable visual display in response to a load measurement on the drive shaft. The load may be measured by a change in current in a motor which drives the shaft. This change in current is then converted to a frequency for variable sound and to a visual display indicative of the load. The load status on the motor may be divided into discrete load levels and correspondingly indicated by discrete levels in the feedback sound frequency and the visual display. This system may be used to monitor and facilitate crossing vascular total occlusions during percutaneous interventions.

### CROSS REFERENCE TO RELATED APPLICATIONS

This application is a continuation-in-part of U.S. patent application Ser. No. 11/388,251, filed Mar. 22, 2006, which is incorporated by reference herein in its entirety.

### BACKGROUND OF THE INVENTION

The present invention is generally related to medical systems and methods. More specifically, the present invention relates to a controller system and method for providing control feedback during crossing stenosis, partial occlusions, or total occlusions in a patient's body, such as in a body or vessel lumen.

Cardiovascular disease frequently arises from the accumulation of atheromatous material on the inner walls of vascular lumens, particularly arterial lumens of the coronary and other vasculature, resulting in a condition known as atherosclerosis. Atheromatous and other vascular deposits restrict blood flow and can cause ischemia which, in acute cases, can result in myocardial infarction or a heart attack. Atheromatous deposits can have widely varying properties, with some deposits being relatively soft and others being fibrous and/or calcified. In the latter case, the deposits are frequently referred to as plaque. Atherosclerosis occurs naturally as a result of aging, but may also be aggravated by factors such as diet, hypertension, heredity, vascular injury, and the like.

Atherosclerosis can be treated in a variety of ways, including drugs, bypass surgery, and a variety of catheter-based approaches which rely on intravascular widening or removal of the atheromatous or other material occluding the blood vessel. Particular catheter-based interventions include angioplasty, atherectomy, laser ablation, stenting, and the like. For the most part, the catheters used for these interventions must be introduced over a guidewire, and the guidewire must be placed across the lesion prior to catheter placement. Initial guidewire placement, however, can be difficult or impossible in tortuous regions of the vasculature. Moreover, it can be equally difficult if the lesion is total or near total, i.e. the lesion occludes the blood vessel lumen to such an extent that the guidewire cannot be advanced across the lesion.

To overcome this difficulty, forward-cutting atherectomy catheters have been proposed. Such catheters usually can have a forwardly disposed blade (U.S. Pat. No. 4,926,858) or rotating burr (U.S. Pat. No. 4,445,509). While effective in some cases, these catheter systems, even when being advanced through the body lumen with a separate guidewire, have great difficulty in traversing through the small and tortuous body lumens of the patients and reaching the target site.

Devices for crossing occlusions or stenoses which can access small, tortuous regions of the vasculature and which can remove atheromatous, thrombotic, and other occluding materials from within blood vessels are described in U.S. patent application Ser. No. 11/236,703, filed Sep. 26, 2005, assigned to the assignee of the present application and incorporated herein by reference. While such devices successfully pass through partial occlusions, total occlusions, or stenosis, and are able to macerate blood clots or thrombotic material, further improvements would be advantageous.

### BRIEF SUMMARY OF THE INVENTION

The present invention relates to controller systems and methods for providing control feedback during crossing tight stenosis, partial occlusions, total occlusions, or chronic total occlusions in a patient's body, such as in a body or vessel lumen. The devices for removing occlusive material and passing through occlusions, stenosis, thrombus, plaque, calcified material, and other material in a neuro, coronary, and peripheral body lumens generally include a hollow and elongate member that is advanced through a blood vessel lumen and positioned adjacent the occlusion or stenosis. An occlusive material (e.g., plaque) drilling assembly is positioned at or near a distal tip of the hollow member to create an opening in the occlusion. The plaque drilling assembly generally comprises a drive shaft having a distal tip that is oscillated (e.g., rotated in one direction for a period of time, then rotated in the reversed direction for a period of time), axially reciprocated (e.g., pecking), rotated and/or vibrated and advanced from within an axial lumen of the hollow member. Once the hollow and elongate member has reached the lesion, the hollow member with the exposed oscillating, axially reciprocating, rotating and/or vibrating drive shaft may be advanced into the lesion (or the hollow member may be in a fixed position and the drive shaft may be advanced).

Advantageously, the controller systems of the present invention provide a control unit coupled to the occlusion-crossing device. The control unit has a processor which produces a variable sound in response to a load measurement on the drive shaft, particularly during advancement of the distal tip in the occluded vessel lumen. The load measurement comprises a change in current in a motor which drives the shaft. It will be appreciated however that the load may be measured in a variety of other ways, as for example measuring a change in voltage, amperage, or other electrical signals related to the drive shaft motor, such as monitoring the change of rotational speed of the drive shaft via an encoder within the motor. The drive motor is generally mechanically attachable to a proximal end of the drive shaft to move (e.g., oscillating, axially translating, reciprocating, rotating, vibrating) the drive shaft and distal tip.

This change in current, which accurately measures the load on the drive shaft, is then converted to a variable frequency sound. The sound frequency may comprise a pitch or tone which changes with the measured load on the motor according to a predetermined relationship. For example, as the measured load or resistance encountered increases, the pitch or tone of the sound may also increase. In one embodiment, the volume of the variable sound is constant and the pitch changes as a function of the measured motor amperage. A selection may be made as to whether the pitch of the variable sound increases or decreases with the motor amperage. A selection may also be made as to whether the pitch of the variable sound changes along a continuous spectrum or in discrete steps. A speaker may be electrically coupled to the processor in the control unit for emitting the sound. Further, an audio amplifier may be electrically coupled between the processor and the speaker in the control unit to amplify the sound prior to emission from the speaker. A visual display may also be electrically coupled to the processor to provide visual feedback of the motor amperage level in addition to and corresponding with the audio feedback provided by the variable sound.

The device used to cross an occlusion, which is described in more detail in co-pending U.S. patent application Ser. No. 11/236,703, comprises an elongate hollow deflectable body having an axial lumen. The drive shaft preferably comprises an oscillatory core element which is movably receivable within the axial lumen. That is, the oscillatory core element is received within the axial lumen and movable within the lumen. A handle may further be coupled to a proximal end of the device. The motor preferably resides within a distal end of the device handle and is mechanically secured to avoid any oscillatory or axial movement during operation. Typically, the drive motor is coupled to the control unit via wire leads or cables. The electronic circuitry in the control unit (e.g., processor) controls activation of the motor to oscillate (e.g., polarity, period, time), axially translate, reciprocate, rotate and/or vibrate the drive shaft besides measuring loads and producing variable sounds associated therewith. It will be appreciated that the control unit may optionally be positioned with the drive motor within the handle component of the device.

In another aspect of the present invention, methods for providing control feedback during crossing of an occlusion or stenosis within a vessel lumen are provided. A device configured to cross the occlusion or stenosis, as described above, is positioned into the vessel lumen adjacent the occlusion or stenosis. A drive shaft is activated within an axial lumen of the device. A level of load on the drive shaft is measured. In response to the load measurement, a variable sound and/or a variable visual feedback display are produced. An operating physician may utilize the feedback produced by the controller to guide the advancement of the oscillating drive shaft. For example, the physician may advance the oscillating drive shaft into the occlusion for so long as the feedback produced remains within an acceptable range. By receiving feedback regarding high motor load, the physician may avoid a condition where the drive shaft is stalled or immobilized. By monitoring changes in resistance (as indicated by the accompanying load level), the physician may detect whether the drive shaft is outside the vessel lumen and within sub-intimal space. Similarly, the physician may also detect whether the drive shaft has crossed the occlusion in the lumen by monitoring a drop in the resistance met by the drive shaft.

Measuring a load comprises measuring a change in current in a motor which drives the shaft. Typically, the load on the drive motor is expressed in milliamps and varies according to the resistance encountered by the drive shaft, particularly its distal tip, in the occluded vessel lumen. The load on the motor may be detected through the measurement of voltage across a known resistor which is directly proportional to the current flowing through the resistor. The resistor may have a resistance in a range from about 0.1 ohms to about 10 ohms. For example, two 1 ohm resistors may be provided for an oscillatory drive shaft, one resistor for each direction of the oscillatory drive motor. The amperage related voltage is then compared to a reference voltage, which may be in a range from about 0.2 volts to about 1.0 volts, as for example 0.53 volts.

A variable sound is then produced by converting the change in current (i.e., the difference between the measured load and the reference voltage) to a frequency for sound. The sound comprises a pitch or tone which may be substantially proportional to the measured load on the motor. For example, the larger the difference between the measured load and the reference voltage, the larger the change in the pitch or tone of the sound. The different audible tones of the sound are noticeable due the change of the frequency of the signal generated by change in the electrical signal of the load measurement. Hence, the sound, which is expressed in hertz, varies according to the resistance encountered by the drive shaft as measured by the current change in the drive motor.

The sound frequency may be in a range from about 30 hertz to about 10,000 hertz, preferably in a range from about 50 hertz to about 5,000 hertz and indicate a variety of load conditions encountered within the vessel lumen. In particular, the control unit may provide higher to lower pitches depending on the resistance encountered by the motor through the drive shaft distal tip. For example, the variable sound may have an increasing or high pitch or tone so as to indicate a high level of occlusion in the vessel lumen. In this instance, the drive shaft distal tip is encountering high resistance so as to indicate a high level of calcification inside the vessel lumen. Alternatively, the variable sound may have a decreasing or low pitch or even no pitch (e.g., no sound) when no load is detected. The electronic circuitry (e.g., processor) in the control unit may further automatically disable the occlusion-crossing device in this situation for safety purposes.

The pitch of the feedback sound may change continuously as a function of the motor load or may change in discrete steps, corresponding to previously pre-selected threshold load levels. A visual feedback display may also be provided in the control unit to visually indicate the level of resistance met by the distal tip of the drive shaft and corroborate the audio feedback sound. An option may also be provided as to whether the pitch of the feedback sound should increase or decrease as a function of motor amperage.

The methods may further comprise emitting the sound from a speaker. Optionally, the sound may be amplified prior to emission from the speaker via an audio amplifier which may additionally be adjustable. As described above, activating may comprise oscillating the drive shaft. Oscillation of the drive shaft may further comprise changing polarity after a period of time in a range from about 0.3 seconds to about 1.2 seconds, preferably in a time range of about 0.7 seconds. The activating, measuring, producing, and changing polarity steps are carried out by electronic circuitry (e.g., processor) in the control unit.

A further understanding of the nature and advantages of the present invention will become apparent by reference to the remaining portions of the specification and drawings.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIG. 1 illustrates an exemplary controller system constructed in accordance with the principles of the present invention.

FIG. 2 illustrates an exploded view of a distal end portion of the device of FIG. 1 comprising a drive shaft disposed within a hollow, deflectable body.

FIG. 3 is a simplified block diagram illustrating the controller system of the present invention.

FIG. 4 is a simplified flow diagram illustrating a method for providing control feedback during crossing of an occlusion or stenosis within a vessel lumen in accordance with the principles of the present invention.

FIGS. 5 through 8 illustrate exemplary electrical schematic drawings for electronics that can be used in an embodiment of the present invention.

FIG. 9 illustrates the frequency of the audio feedback changing continuously as a function of motor load.

FIG. 10 illustrates the frequency of the audio feedback changing in discrete steps as motor load increases.

FIG. 11 illustrates the frequency of the audio feedback changing in discrete steps as motor load increases.

### DETAILED DESCRIPTION OF THE INVENTION

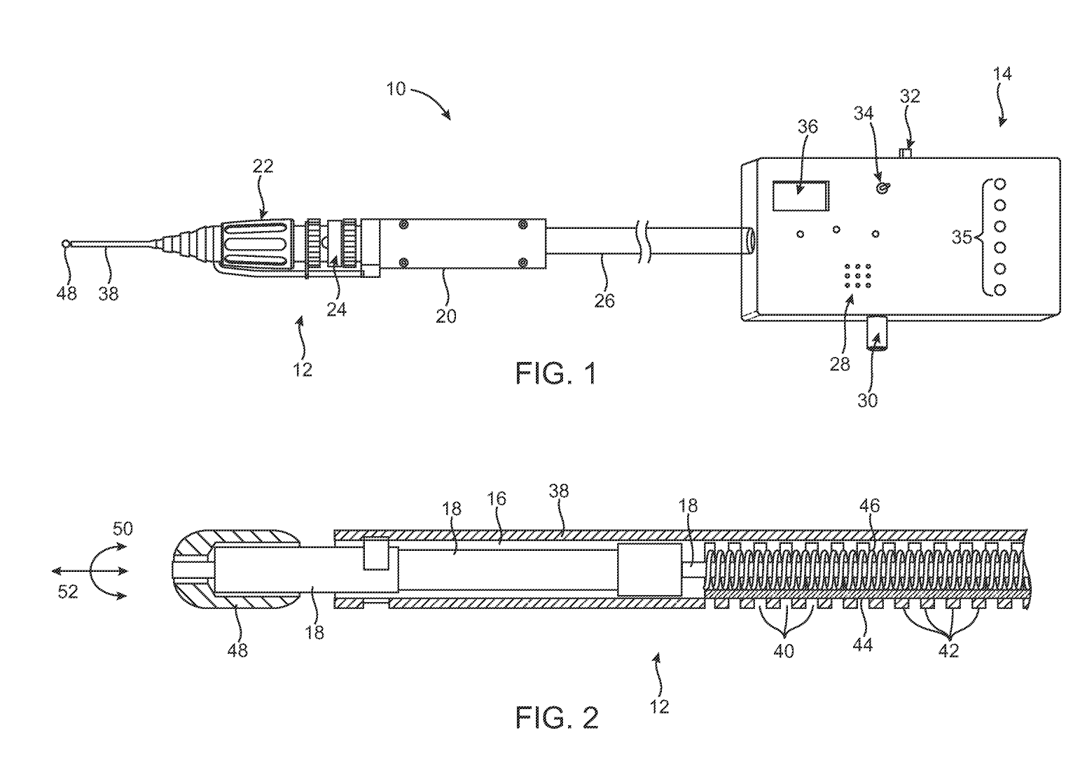

Referring now to FIG. 1, an exemplary controller system 10 constructed in accordance with the principles of the present invention is illustrated. The controller system 10 includes a device 12 configured to cross an occlusion and a control unit 14. The occlusion-crossing device 12 has an axial lumen 16 and a drive shaft 18 extending through the axial lumen 16, as best shown in FIG. 2. A handle 20 having a torquer knob 22 to torque the device 12 and a deflection wheel 24 to deflect the device 12 may further be coupled to a proximal end of the device 12. The control unit 14 is coupled to the device handle 20 via wire leads or cables 26. The control unit 14 includes a speaker 28, a volume control knob 30 which may adjust audio amplification, a main on/off power supply switch 32, a momentary switch 34, a display comprising a linear array of light emitting diodes (LED) 35, and a timer liquid crystal display (LCD) 36. The control unit may have a length in a range from about 5 cm to about 25 cm, preferably 17 cm, a width in a range from about 5 cm to about 12 cm, preferably 8.5 cm, and a depth in a range from about 1 cm to about 8 cm, preferably 3.5 cm. It will be appreciated that the above depictions are for illustrative purposes only and do not necessarily reflect the actual shape, size, or dimensions of the controller system 10. This applies to all depictions hereinafter.

Referring now to FIG. 2, the occlusion-crossing device 12 of the present invention has steerability, deflectability, flexibility, pushability, and torqueability to be advanced through the tortuous blood vessel without the use of a separate guidewire or other guiding element. Additionally, the device 12 may be sized to fit within an axial lumen of a distal support or access catheter system (not shown), which is described in more detail in U.S. patent application Ser. No. 10/864,075, filed Jun. 8, 2004, assigned to the assignee of the present application and incorporated herein by reference. The distal support catheter system can be delivered either concurrently or sequentially with the advancement of the device 12 to the target site. The position of the catheter system can be maintained and stabilized while the device 12 is advanced.

The occlusion-crossing device 12, which is described in more detail in co-pending U.S. patent application Ser. No. 11/236,703, comprises an elongate hollow deflectable body 38 having a proximal portion, a deflectable distal portion, and a flexible intermediate portion along a length therebetween. In one embodiment, the distal portion of the elongate body 38 may have a fixed, preset deflection. The elongate hollow body 38 movably receives the drive shaft 18 within its axial lumen 16 and is coupled to the handle 20 on the proximal portion. That is, the draft shaft 18 is received within axial lumen 16 and is movable therein. The elongate hollow body 38 may be composed of a unitary structure, such as a single hypotube, which forms a plurality of sections. In a preferred embodiment, the sections comprise a variety of patterns including a proximal interrupted helical pattern and a distal ribbed pattern 40, 42. The elongate hollow body 38 may be formed from a variety of materials, including stainless steel, polymer, carbon, or other metal or composite materials. The body 38 may have an outer diameter in a range from about 0.010 inch to about 0.040 inch, an inner diameter in a range from about 0.005 inch to about 0.036 inch, and a working length in a range from about 150 cm to about 190 cm, as for example in FIG. 1 the length is illustrated as approximately 160 cm.

Referring back to FIG. 2, an exploded view of the distal end portion of the device 12 shows the drive shaft 18, a tapered pull tube 44 for deflection via the deflection wheel 24, and a radiopaque coil 46 for aid in viewing under fluoroscopy, which are all disposed within the axial lumen 16 of the elongate hollow body 38. The drive shaft 18 may be movably or fixedly disposed at the distal end of the elongate hollow body 38. A distal tip 48 of the drive shaft 18 extends distally of the distal end of the hollow body 38. Upon activation, the distal tip 48 of the drive shaft 18 creates a passageway or enlarges a passageway through the occlusion or stenosis within the vessel lumen. Generally, the distal tip 48 of the drive shaft 18 creates a path at least as large as a perimeter of a distal end of the hollow body 38. However, it will be appreciated that the path can also have the same perimeter or smaller perimeter than the distal end of the hollow body 38.

The drive shaft 18 in this embodiment preferably comprises an oscillatory core element, as depicted by arrow 50, which is movably receivable within the axial lumen 16. That is, the oscillatory core element is received within axial lumen 16 and is movable therein. The preferred oscillating operating mode 50 is of particular benefit as it prevents tissue from wrapping around the distal tip 48 of the drive shaft 18. This oscillating rotation (i.e., rotation in one direction for a period of time followed by rotation in the reverse direction for a period of time) allows for enhanced penetration through, in, and/or out of the occlusive or stenotic material. Typically, the drive shaft 18 may be oscillated so that it changes polarity after a period of time. The period of time may in a range from about 0.2 seconds to about 5.0 seconds, preferably in a range from about 0.3 seconds to 1.2 seconds, and more preferably in a range of about 0.7 seconds. Oscillations may be in a range from about 3,600 degrees to about 360,000 degrees.

The drive shaft 18 may additionally comprise an axially translatable drive shaft as depicted by arrow 52 for axial or reciprocation movement so as to completely cross an occlusion. Oscillation movement 50 and reciprocation movement 52 of the drive shaft 18 may be carried out sequentially or simultaneously. Generally, oscillation and/or reciprocation 50, 52 movement of the drive shaft 18 are carried out by a drive motor within the device handle 20, which is described in more detail below. Alternatively, the physician may also manually oscillate and/or reciprocation the drive shaft 18. Additionally, the movable drive shaft 18 may be extended from a retracted configuration to an extended configuration relative to the distal portion of the hollow body 38, wherein the drive shaft 18 is simultaneously or sequentially extended and oscillated.

The drive shaft 18 may be formed from a variety of materials, including nitinol, stainless steel, platinum iridium, and like materials and have a diameter in a range from about 0.003 inch to about 0.036 inch and a working length in a range from about 150 cm to about 190 cm. The drive shaft distal tip 48 will preferably have an outer perimeter which is equal to or larger than a diameter of the hollow body 38 so as to create a path at least as large as a perimeter of the distal end of the body 38. As can be appreciated, the diameter of the drive shaft 18 will depend on the dimension of the inner lumen 16 of the hollow body 38, the pull tube 44, and/or the radiopaque coil 46.

As mentioned above, the hollow device 12 of the present invention may have steerability, deflectability, flexibility, pushability, and torqueability which allow it to be positioned through the tortuous blood vessel. Once properly positioned adjacent the occlusion or stenosis, the distal tip 48 of the drive shaft 18 is oscillated and simultaneously or sequentially advanced into the occlusion or stenosis in the vessel lumen to create a path in the occlusion or stenosis. It will be appreciated that the hollow body 38 and/or the drive shaft 18 may be advanced to create a path through the occlusion or stenosis. For example, once the hollow body 38 has reached the occlusion, the body 38 together with the oscillating drive shaft 18 may be advanced into the occlusion. Alternatively, the body 38 may be in a fixed position and only the oscillating drive shaft 18 may be advanced into the occlusion.

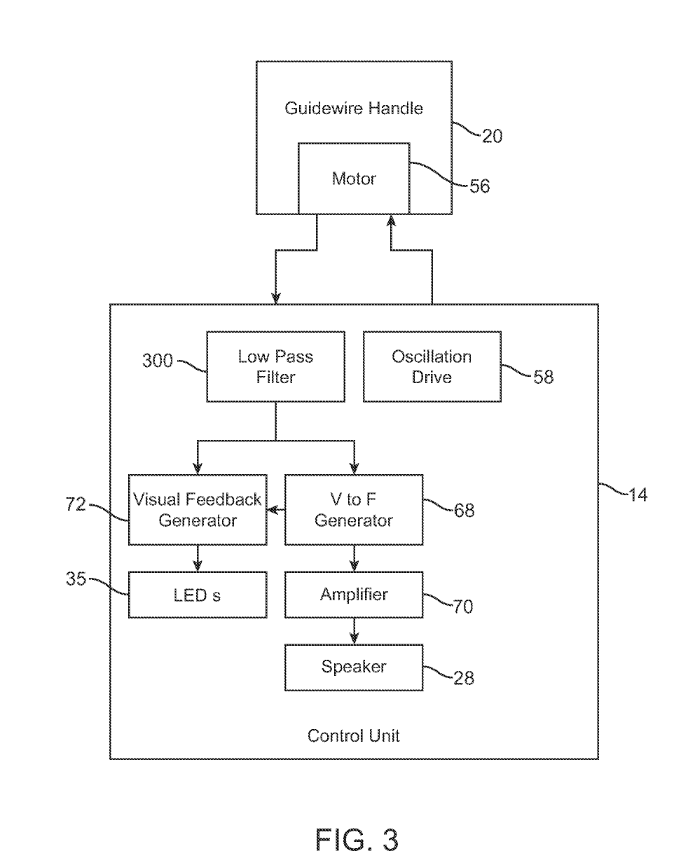

Referring now to FIG. 3, a simplified block diagram of the controller system 10 of the present invention is illustrated. The control unit 14 may include a low pass filter 300 which removes noise from the motor current signal received from motor 56 and may also provide amplification of the signal. The control unit 14 may also include a V to F generator 68 which produces a variable sound in response to a load measurement (e.g., resistance encountered) on the drive shaft 18, particularly during advancement of the distal tip 48 in the occluded vessel lumen. Generator 68 may be a microprocessor, one or more discrete logic blocks or the like. The load or resistance encountered may be measured by a change in current in a motor 56 which drives the shaft 18, which may be measured by a change in voltage across a feedback resistor. The motor 56 preferably resides within the device handle 20 and is mechanically attachable to a proximal end of the drive shaft 18 to move (e.g., oscillate, axially translate, reciprocate, rotate, vibrate) the drive shaft 18 and distal tip 48. Typically, the drive motor 56 is electrically coupled to the control unit via the wire leads or cables 26 (FIG. 1). The voltage signal from motor 56 may be directly input into visual feedback generator 72 through filter 300 such that the generator 72 independently provides an appropriate signal to LED display 35 based on the measured voltage across the feedback resistor of the motor. Alternatively, the frequency data generated by V to F generator 68 may be input into generator 72, which may then provide an appropriate signal to LED display 35 based on the frequency data.

The electronic circuitry in the control unit 14, as for example the oscillation system 58, controls activation of the motor 56 to oscillate (e.g., polarity, period, time), axially translate, reciprocate, rotate and/or vibrate the drive shaft 18. For example, the oscillation system 58 may control the output of the oscillation mode 50 of ± and ∓ at 0.7 seconds in each direction of the oscillatory drive shaft 18. This output mode may be provided by activation of the momentary switch 34. As another example, the oscillation system 58 may measure the accumulated oscillation time or oscillation cycles. In this instance, the device 12 may be automatically disabled once the accumulated oscillation time has exceeded a time threshold in the range from about 60 seconds to about 3,600 seconds, as for example 600 seconds. The accumulated oscillation time may be constantly displayed on the LCD display 36 on the control unit 14. The next oscillation interval may be initiated by turning the main power switch 32 off and then back on.

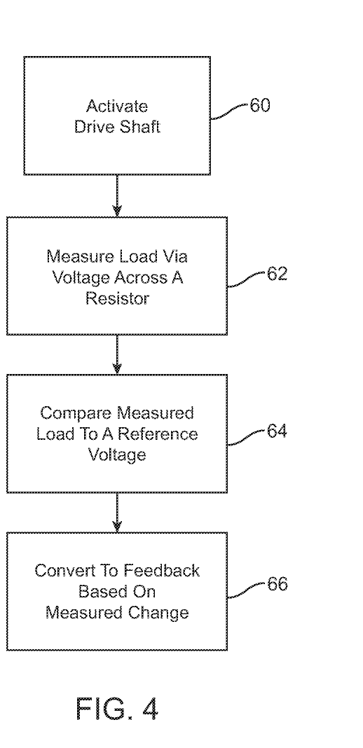

Referring now to FIG. 4, the processor 54 in the control unit 14 further measures loads and produces variable sounds and visual feedback associated therewith after activating the drive shaft 18 as depicted by block 60. Measuring a load comprises measuring a change in current in the motor 56 which drives the shaft 18. Typically, the load on the drive motor 56 varies according to the resistance encountered by the drive shaft 18, as for example hard or soft stenosis in the vessel lumen. The load on the motor 56 may be measured through the detection of voltage across a known resistor, as depicted by block 62, which is directly proportional to the current flowing through the resistor. For example, two 1 ohm resistors may be provided for an oscillatory drive shaft 18, one resistor for each direction of the oscillatory drive motor 56. The amperage related voltage is then compared to a reference voltage, as for example 0.53 volts, as depicted by block 64.

As depicted by block 66, feedback is then produced by converting the change in current (i.e., the difference between the measured load and the reference voltage) to a frequency for sound via a voltage to frequency generator 68 (FIG. 3) and a visual display via visual feedback generator 72 (FIG. 3). The sound comprises a pitch or tone which may vary with the measured load on the motor 56 in accordance with a predetermined frequency/load curve. In one example, at least a portion of the frequency/load curve provides for a substantially linear relationship between the sound frequency and the load. As such, the larger the difference between the measured load and the reference voltage, the larger the change in the pitch or tone of the sound. In another example, the frequency may change as a non-linear function of the load. The different audible tones of the sound are noticeable due the change of the frequency of the signal generated by change in the electrical signal of the load measurement. In one approach, the motor current may be tracked through a period within an oscillation cycle. The current is then held for a pre-set period while the motor changes polarity. The hold will typically last longer than the period for the motor to change polarity, but less than the oscillation time of one cycle. This feature may also be referred to as “track and hold,” the purpose of which is to eliminate frequency changes related to the motor reversing directions, which affects the motor current but is not related to the resistance the distal tip is encountering.

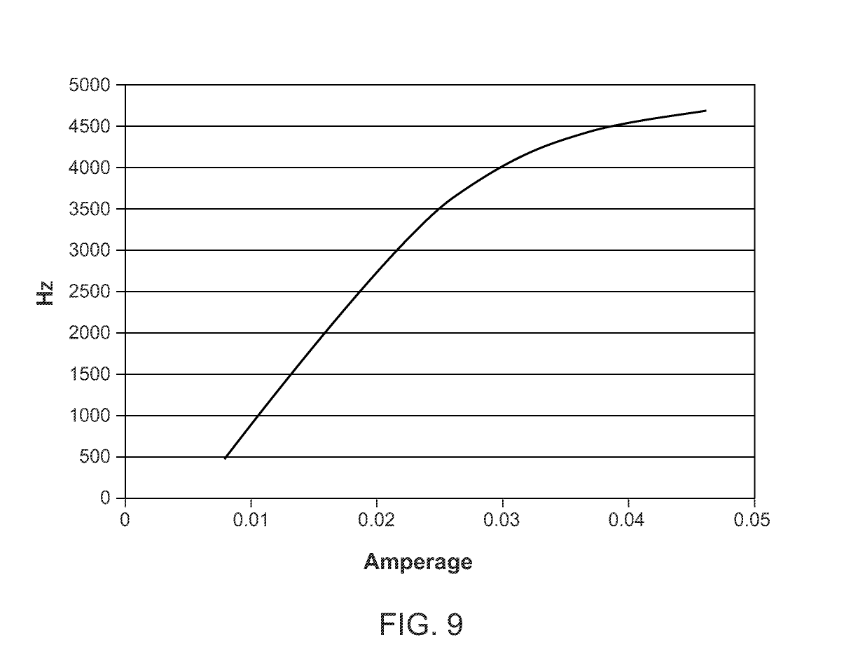

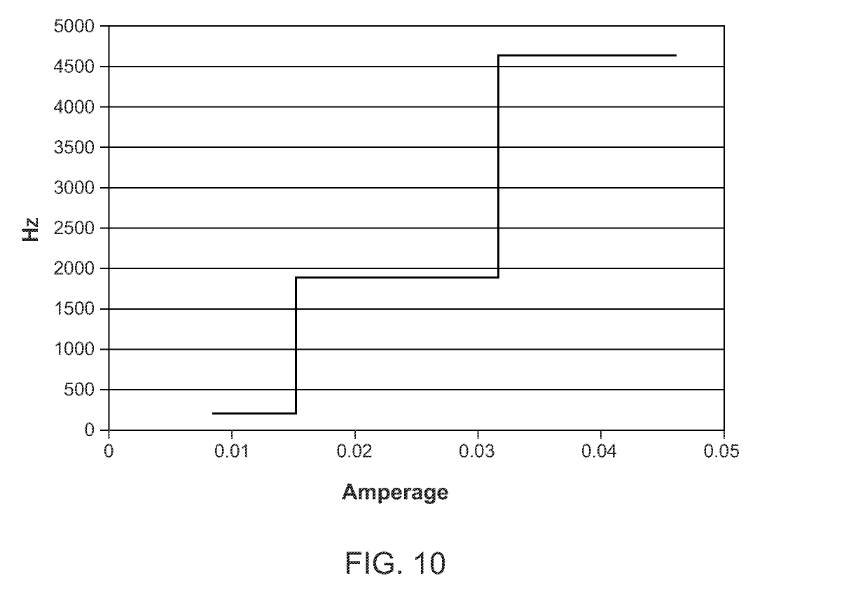

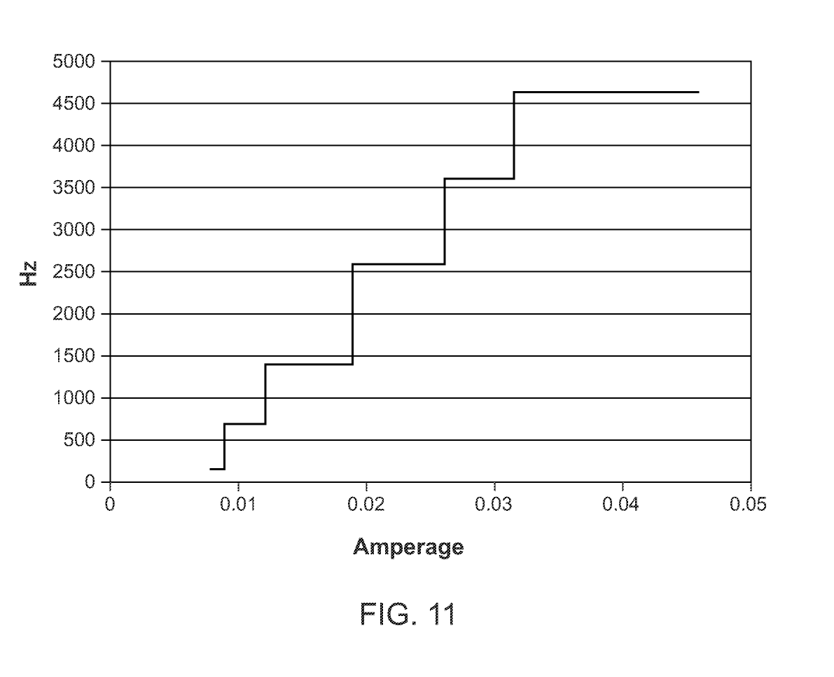

In one embodiment of the invention, the variable sound produced by the control unit is of a constant volume and varies in pitch with the motor amperage or load level. In particular, the pitch of the feedback sound may be within a frequency range, for example, between 50 to 5,000 hertz, that corresponds to the operating range of the motor load (i.e., from the zero load state to the maximum load, immobilized state). In one aspect of the invention, the control unit may be hardwired or programmed to either increase or decrease the frequency with increasing load level. In another aspect of the invention, the control unit may be hardwired or programmed to either change the frequency of the feedback sound in a continuous manner or in a number of discrete steps. As illustrated by FIG. 9, in a first continuous mode, the frequency of the feedback sound is a continuous function of the amperage load on the motor. That is, the frequency/load curve illustrated in FIG. 9 determines the frequency of the feedback sound emitted at any given motor load. It should be noted that at least a portion of the frequency/load curve (e.g., between 0.01 amperes and 0.02) provides a substantially linear relationship between the frequency and the load. FIG. 10 illustrates an alternative approach, in which the frequency of the feedback sound changes in discrete steps. In the arrangement illustrated by FIG. 10, the number of discrete frequency steps is three. However, it should be understood that more or less discrete steps may be used to carry out this aspect of the invention. For example, FIG. 11 illustrates an arrangement with six discrete frequency steps.

The discrete load level steps (that trigger a step change in the feedback frequency) and the frequency steps themselves may or may not be equal steps. In some applications, a substantially linear relationship between frequency and load may be desired, while in other applications, a non-linear relationship may be preferred. For example, based upon clinical experience, the control unit may be hardwired or programmed to alert a user to smaller changes in load level when the load level is within particularly sensitive ranges.

In one embodiment of the invention, the discrete audio steps may be distinguished by changes in the feedback sound other than in its frequency. For example, at one load level the feedback sound may be a constant tone while at another load level the feedback sound may be a beeping sound. This will help the operator identify changes in load independent of frequency changes, which may be difficult for some operators to ascertain. In one embodiment, the control unit may enable the operator to select the type of change desired in the feedback sound.

In another aspect of the invention, a visual feedback display may be provided to indicate the load level on the motor and correspond with or corroborate the audio feedback. This may be accomplished in a number of alternative approaches. For example, FIG. 1 illustrates one illustrative embodiment of the control unit in which the LED display 35 includes a series of individual light elements that indicate the load level on the motor. In one approach, the particular element to be illuminated is determined by the load level on the motor. For example, when signals received from processor 54 (FIG. 3) indicate that the motor is experiencing its lowest load level (which may be the zero load state), a bottom-most light element or no light element may be illuminated. As load levels increase, progressively higher light elements may be illuminated, ending with a top-most light element being illuminated at the highest load level (which may be the frozen tip state in which the distal tip is immobilized). In another approach, higher levels of load are indicated by the increasing number of contiguous light elements that are illuminated. In yet another approach, the light elements are colored light elements, whereby the color of the elements are different and may be chosen from a continuous color spectrum (e.g., green to yellow to red) to indicate increasing levels of load. In one preferred approach, two colors of LEDs are used (e.g., green and red) to indicate an operating zone (e.g., green LEDs are lit) and a danger zone (e.g., red LED is lit). It should be understood that a multitude of alternative approaches are available to visually indicate the motor load level, including the use of numerical read outs and other visual indicators. Moreover, the visual feedback output may be displayed externally from the control unit, for example, as a corresponding signal on one or more external display monitors connected to the control unit.

The load levels embraced by the visual feedback display may be the same as the load levels that trigger the discrete feedback levels (e.g., frequency levels) in the audio feedback. For example, if six frequency levels are provided in the audio feedback, then six visual feedback levels may be provided via six light elements. In this embodiment, the audio feedback and visual feedback reinforce each other to provide the user with instinctive feedback. In another embodiment, either the audio feedback or the visual feedback may have fewer levels. For example, the visual feedback may contain six LEDs that are capable of indicating a finer gradation of motor load than the audio feedback, which may only contain three feedback levels to enable ease in discernment by the operator. Moreover, it should be understood that the audio feedback and visual feedback may be provided together when only one form of feedback or neither form of feedback uses discrete load level steps. For example, the audio feedback frequency may be varied continuously with load while the visual feedback may be provided in the form discrete load level steps. Other permutations are contemplated and well within the scope of the present invention.

The combined-audio and visual feedback described above may indicate a variety of load conditions within the vessel lumen. In particular, the control unit 14 may provide appropriate feedback depending on the resistance encountered by the motor 56 through the drive shaft distal tip 48. For example, the variable feedback may indicate that the drive shaft distal tip 48 is encountering low resistance, which in turn indicates a soft plaque inside the vessel lumen. In another example, the variable feedback may show a high load condition which may indicate that the device 12 is encountering a hard, highly calcified occlusion. By monitoring changes in the variable feedback, the physician may also detect whether the drive shaft distal tip 48 is outside the vessel lumen and within sub-intimal space and whether the distal tip 48 has crossed the occlusion in the lumen. For example, by monitoring a decrease in the load state of the drive motor, the physician may detect that the distal tip 48 has crossed the occlusion in the lumen.

In one embodiment, a no-load condition may be indicated in which the volume of the feedback sound may be of a much lower intensity or may not even be noticeably present. Alternatively, the volume of the feedback sound may be constant and only the pitch of the sound indicates load levels. A feedback sound having a constant, unchanging pitch may indicate that the device 12 is non-operational. In this instance, the constant and unchanging no load measurement may indicate a break or fracture in the drive shaft 18. In this situation, all that is audible may be the clicks as the motor 56 changes direction for an oscillatory drive shaft 18. The processor 54 in the control unit 14 may further automatically disable the device 12 in this situation for safety purposes. Moreover, as further safety features, the DC current from the motor may be disconnected by the control unit 14 if the following failure conditions occur: i) oscillating time in one direction is greater than a maximum allowed output time, and ii) there is no reverse direction between two identical oscillation cycles.

An operating physician may utilize the feedback produced by the controller to guide the advancement of the oscillating drive shaft. For example, the physician may advance the oscillating drive shaft into the occlusion for so long as the feedback produced remains below a threshold level (i.e., indicating that the load on the drive motor remains below a threshold level). By receiving feedback regarding high motor load, the physician may avoid a condition where the drive shaft is stalled or immobilized within the occlusion. Similarly, the physician may also use the feedback to advance the oscillating drive shaft into the occlusion so long as the feedback remains above a threshold level (i.e., indicating that the motor load is above a threshold level). Therefore, by monitoring the feedback produced by the controller, an operating physician may keep the motor within an acceptable operating range and therefore avoid conditions, for example, where the drive shaft becomes immobilized or passes outside of the lumen. Moreover, the physician may also use the feedback data to determine the nature of the tissue being encountered by the drive shaft and ascertain the position of the drive shaft within the occlusion.

In one illustrative arrangement with six levels, the baseline feedback level 1 (e.g., lowest pitch feedback sound and zero or one illuminated light element) may be set to indicate motor load of about 0 to 10% and the highest feedback level 6 (e.g., highest pitch feedback sound and all light elements illuminated) may be set to indicate motor load of about 90 to 100%. Feedback level 5 may be set to indicate the typical load of about 75 to 90% measured when hard calcified tissues are encountered, for example, when crossing the proximal and distal caps at the entry and exit segments of chronic total occlusions. Feedback level 4 may be set to indicate the load of about 50 to 75% typically encountered with fibrous-calcified tissue within the occlusion between the proximal and distal caps. Feedback level 3 may be set to indicate the load of about 25 to 50% typically encountered with collagenous tissue between the proximal and distal caps. Feedback level 2 may be set to indicate the load of about 10 to 25% typically encountered with organized thrombus within the occlusion between the proximal and distal caps. It should be understood that the foregoing represents only one possible arrangement of the present invention and that other arrangements are fully contemplated, especially as clinical experience is accumulated.

The feedback information described in the foregoing description is valuable to physicians in their attempts to cross CTO lesions while staying within the true vessel lumen and avoiding the advancement of the device into the sub-intimal space. Penetrating through the sub-intimal space creates a “false lumen” and may be undesirable as it may lead to potential bleeding complications, dissections, or vessel wall internal tears that may significantly complicate the completion of CTO interventional procedures. However, in certain cases, it may be acceptable to advance into the sub-intimal space and the feedback information of the present invention may be used to guide physicians in such procedures as well.

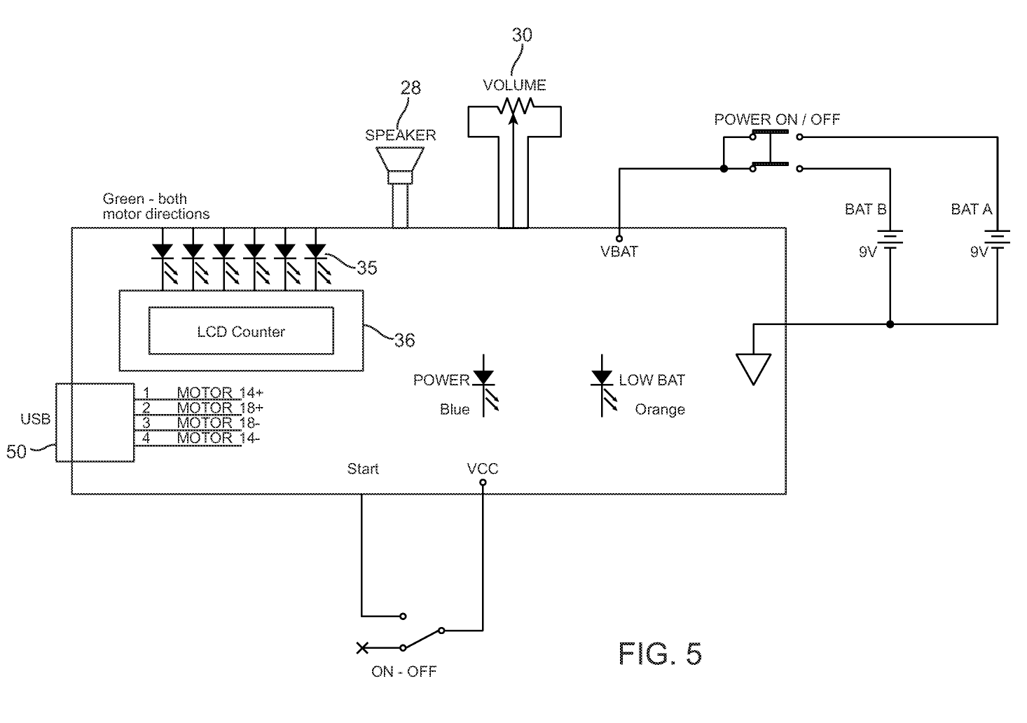

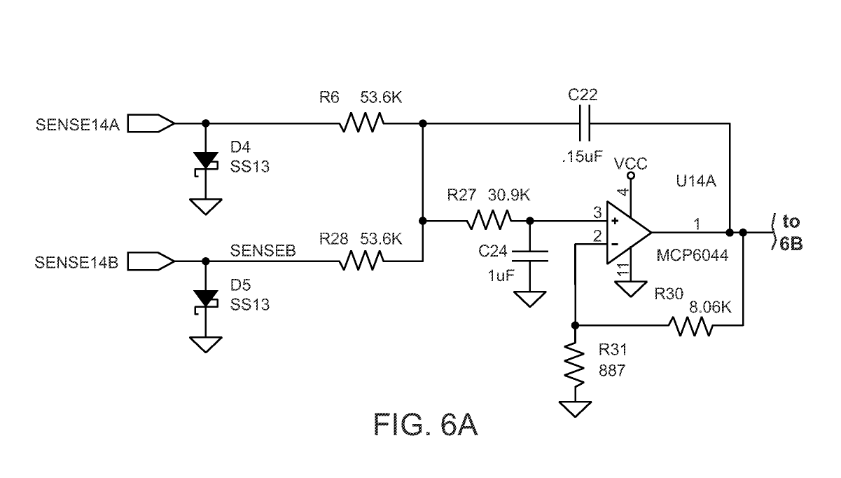

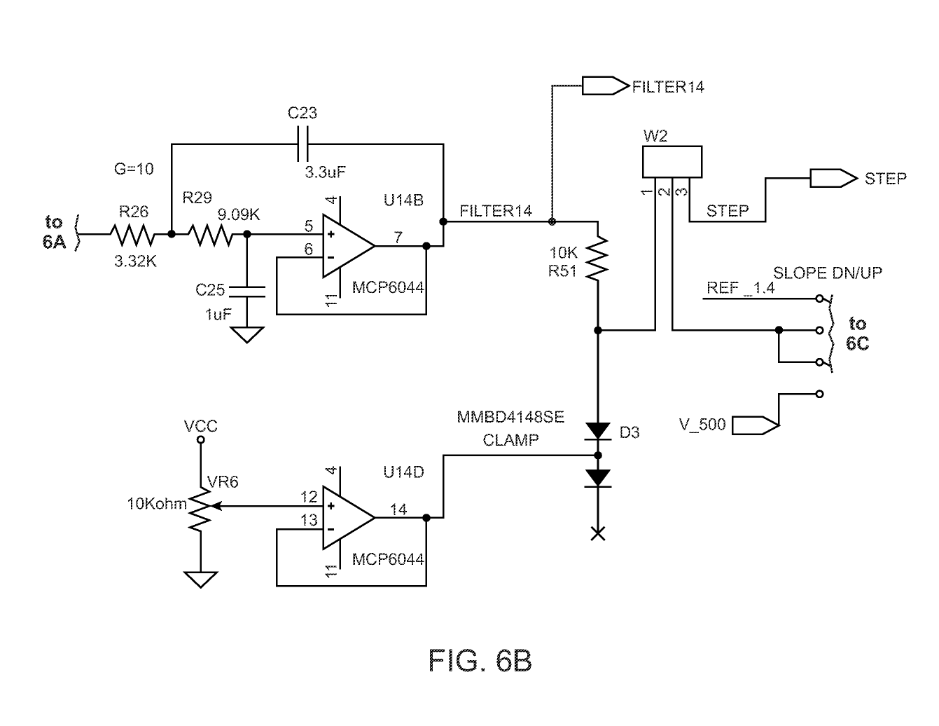

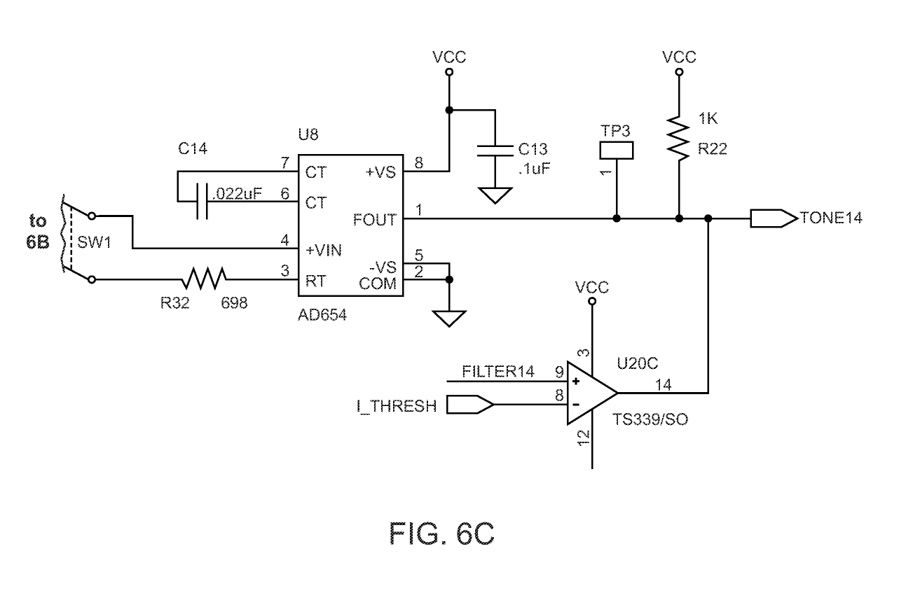

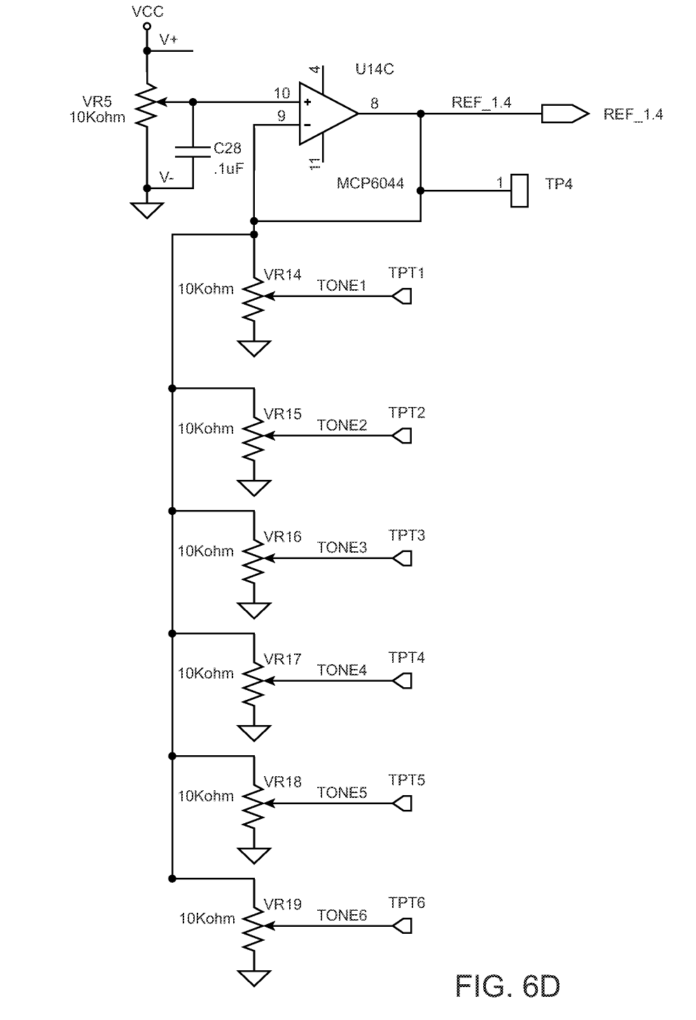

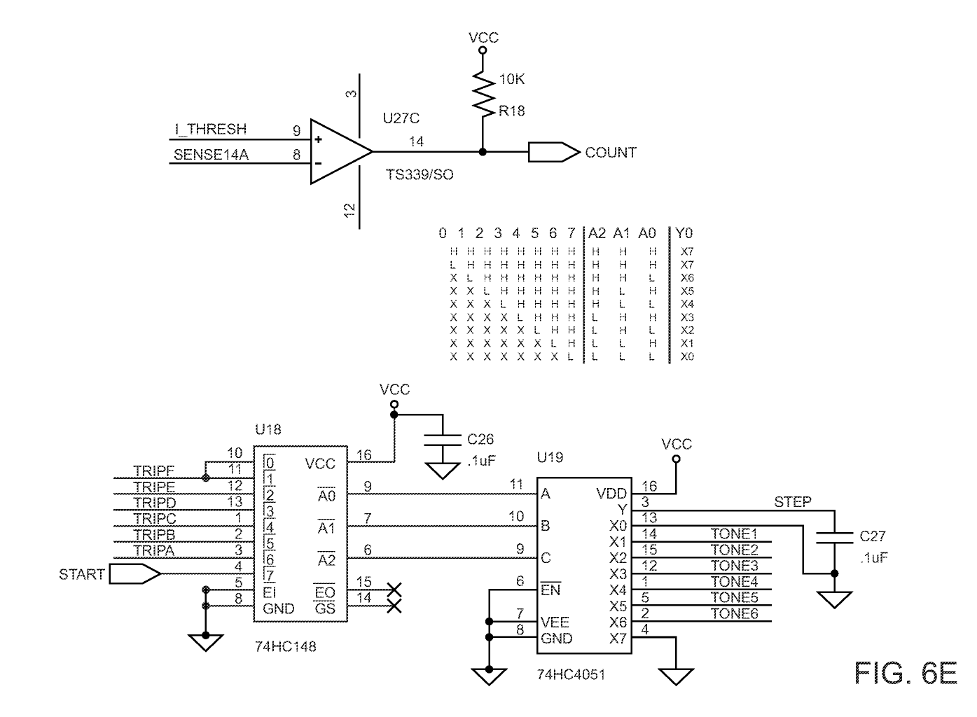

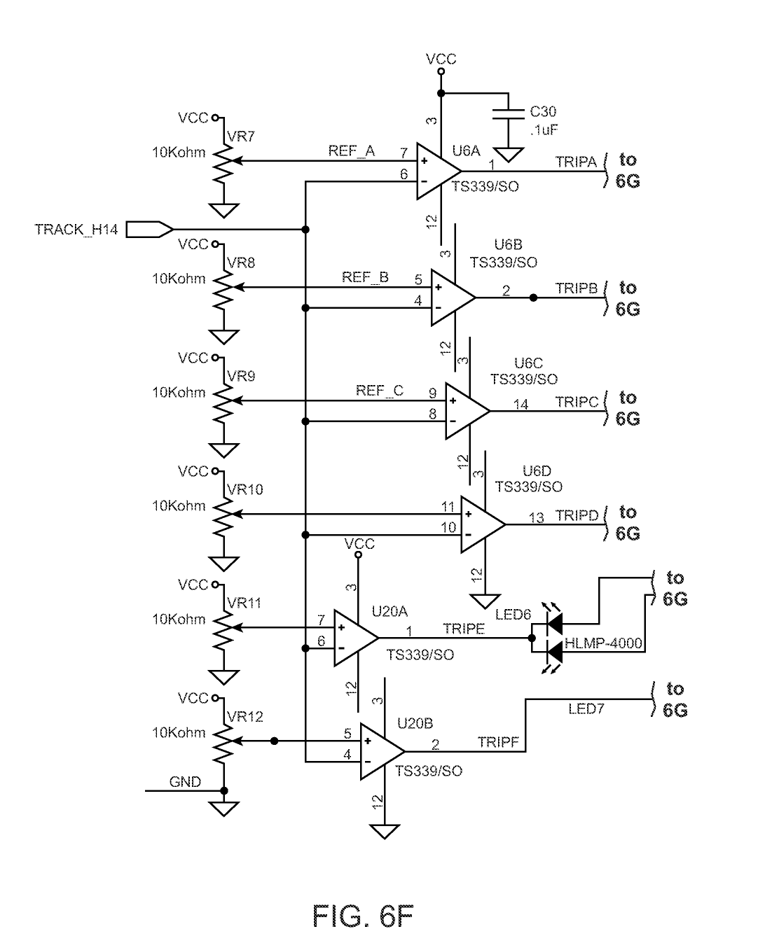

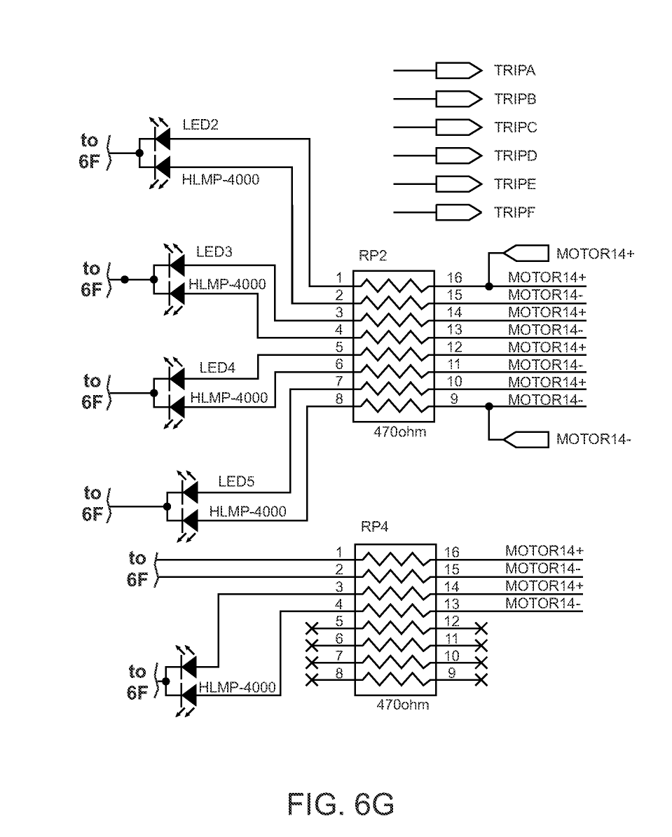

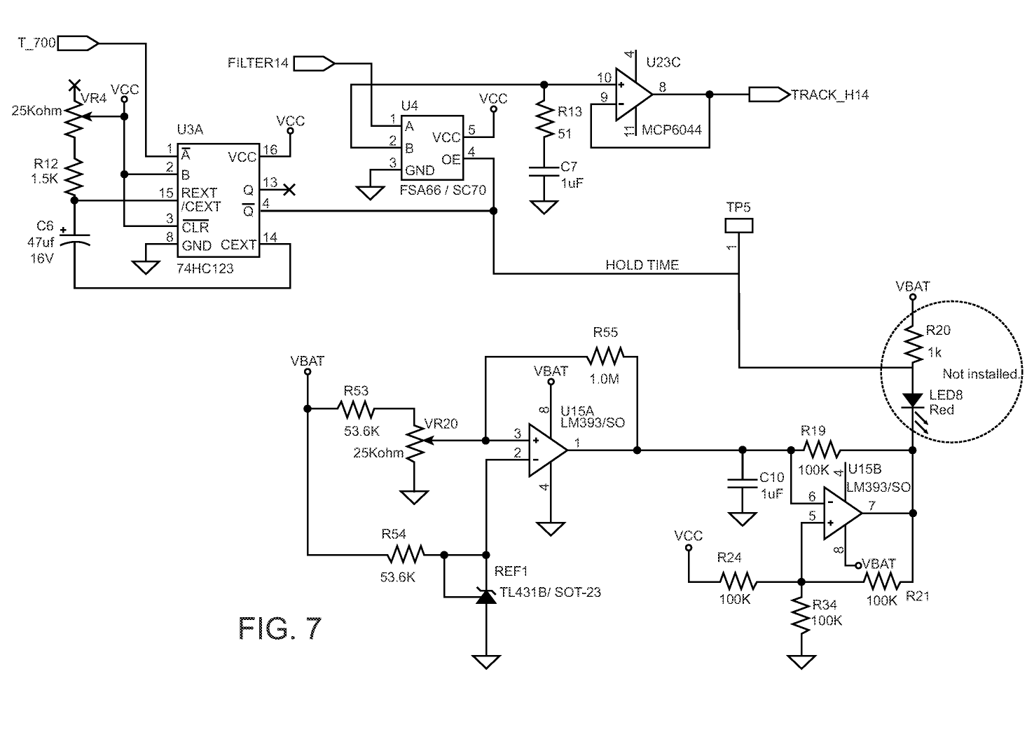

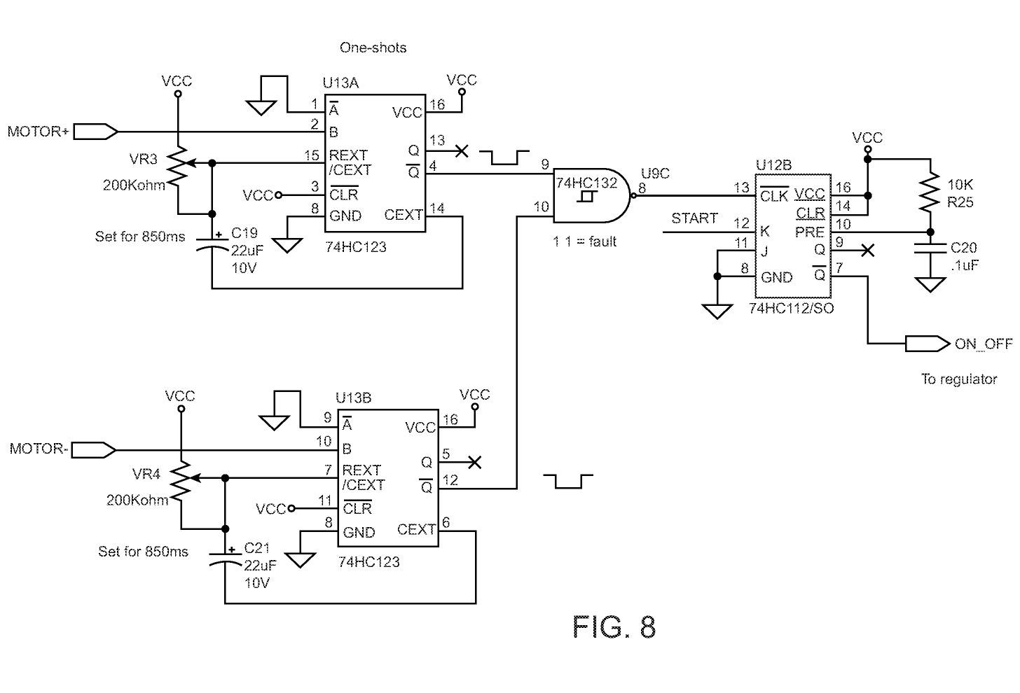

FIGS. 5 through 8 show exemplary electronic circuit diagrams of a circuitry implementation that can be used within the control unit 14 of the present invention. It is understood that many other circuit implementations can be used and yet still arrive at embodiments of the invention. FIG. 5 illustrates various components of the control unit 14 including the speaker 28 for emitting sound, the volume control knob 30 for adjusting amplification of the audio amplifier 70 (FIG. 3), the LCD counter 36, LEDs 35, and USB port 50. In one embodiment, the control unit 14 may include two or more independent internal circuits dedicated to controlling specified models of occlusion-crossing devices. For example, control unit 14 may recognize a particular model of an occlusion-crossing device connected to the USB port 50 and use the appropriate internal circuit to control the device. Many commercially available processors may be used in controller 14, including 20 MHz processors commercially available from Microchip Inc., discrete logic blocks, or the like. The control unit 14 may be powered by two 9V alkaline batteries via the main on/off power supply switch 32. The power supply may further include a voltage regulator which allows for adjustment of optimum motor 56 speed and torque. It will further be appreciated that the control unit 14 may alternatively be powered via voice activation, wireless activation, or Bluetooth® footswitch technology in lieu of manual activation with switch 32. FIG. 6 illustrates a sample circuit of the voltage to frequency generator 68 and a sample circuit of the visual feedback circuitry using dual color LEDs (e.g., green and red). FIG. 7 is a sample circuit for implementing the “track and hold” feature of the present invention that also includes circuitry for the low battery LED shown in FIG. 5. FIG. 8 is a sample circuit for implementing the safety shut down feature of the present invention.

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. For example, as described above, another way to measure load is by reading the rotational speed (e.g., rotations per minute) of the drive shaft using an encoder within the drive motor. Basically, the encoder reads the number of revolutions that the drive shaft is rotating at and when any load is sensed then sound changes proportionally. In another example, it is fully contemplated that the present invention may be implemented as a separate control unit as shown in FIG. 1 or may be incorporated into a handle of the occlusion-crossing device. Therefore, the above description should not be taken as limiting the scope of the invention which is defined by the appended claims.

What is claimed is:

1. A driver for a device having a drive shaft, said driver comprising:
a motor coupled to the drive shaft configured to rotate and/or oscillate the drive shaft;
a load sensor coupled to the motor having an output representative of load on the motor; and
at least one of an audible and visual output coupled to the sensor output and providing load status to a user.

2. The driver of claim 1, wherein the load status is represented as a plurality of discrete load levels.

3. The driver of claim 2, wherein there are at least two discrete load levels.

4. The driver of claim 1, wherein the audible output changes in frequency as the load on the motor increases.

5. The driver of claim 4, wherein the frequency increases as the load increases.

6. The driver of claim 4, wherein the frequency decreases as the load increases.

7. The driver of claim 1, wherein the audible output is of a constant volume.

8. The driver of claim 4, wherein the change in frequency is a continuous function of the load.

9. The driver of claim 2, wherein the audible output undergoes step changes in frequency corresponding with the plurality of discrete load levels.

10. The driver of claim 1, wherein the audible output and visual output are simultaneously provided.

11. The driver of claim 2, wherein the visual output indicates one of the plurality of discrete load levels.

12. The driver of claim 2, wherein the visual output comprises a plurality of light elements corresponding to the plurality of discrete load levels.

13. The driver of claim 2, wherein the visual output comprises a corresponding signal displayed on a monitor external to the driver.

14. The driver of claim 1, wherein current to the motor is disconnected by the driver if at least one condition occurs from the group consisting of: i) oscillating time in one direction is greater than a maximum allowed output time, and ii) there is no reverse direction between two identical oscillation cycles.

15. The driver of claim 1, wherein the load sensor tracks the load on the motor through a period within an oscillation cycle of the motor and then enters a hold period while the motor changes polarity.

16. A method for crossing a total occlusion, said method comprising:
engaging a rotating and/or oscillating shaft against the occlusion;
providing at least one of an audible and visual feedback indicative of load produced on a motor driving the shaft; and
advancing the shaft into the occlusion for so long as the feedback produced remains below a threshold level.

17. The method of claim 16, further comprising advancing the shaft into the occlusion for so long as the feedback remains above a second threshold level.

18. The method of claim 16, wherein the threshold level is indicative of a substantially maximum load on the motor.

19. The method of claim 17, wherein the second threshold level is indicative of substantially no load on the motor.

20. The method of claim 16, wherein the providing step comprises providing feedback represented as a plurality of discrete load levels.

21. The method of claim 20, wherein the plurality of discrete load levels comprises at least two discrete load levels.

22. The method of claim 16, wherein providing an audible feedback comprises providing a feedback sound that changes in frequency as the load produced on the motor increases.

23. The method of claim 22, wherein providing a feedback sound that changes in frequency as the load produced on the motor increases comprises increasing the frequency of the feedback sound as the load increases.

24. The method of claim 22, wherein providing a feedback sound that changes in frequency as the load produced on the motor increases comprises decreasing the frequency of the feedback sound as the load increases.

25. The method of claim 16, wherein providing an audible feedback comprises providing a feedback sound of a constant volume.

26. The method of claim 22, wherein providing a feedback sound that changes in frequency as the load produced on the motor increases comprises changing the frequency as a continuous function of the increase in the load.

27. The method of claim 20, wherein providing feedback represented as a plurality of discrete load levels comprises providing a feedback sound that undergoes step changes in frequency corresponding with the plurality of discrete load levels.

28. The method of claim 16, wherein providing at least one of an audible and visual feedback indicative of load produced on a motor driving the shaft comprises providing the audible and visual feedback simultaneously.

29. The method of claim 20, wherein providing feedback represented as a plurality of discrete load levels comprises providing a visual display that indicates one of the plurality of discrete load levels.

30. The method of claim 20, wherein providing feedback represented as a plurality of discrete load levels comprises providing a plurality of light elements corresponding to the plurality of discrete load levels.

31. The method of claim 16, wherein providing the visual feedback comprises displaying a signal on a monitor.

32. The method of claim 16, further comprising disconnecting current to the motor if at least one condition occurs from the group consisting of: i) oscillating time in one direction is greater than a maximum allowed output time, and ii) there is no reverse direction between two identical oscillation cycles.

33. The method of claim 16, further comprising:
tracking the load produced on the motor, using a load sensor, through a period within an oscillation cycle of the motor; and
placing the load sensor into a hold period while the motor changes polarity.
